(19) United States
(12) Patent Application Publication
(10) Pub. No.: US 2023/0340450 A1
(43) Pub. Date: Oct. 26, 2023

(54) AN ENZYME-POLYMER MATRIX

(71) Applicant: STELLENBOSCH UNIVERSITY, Stellenbosch (ZA)

(72) Inventors: Bianke Loedolff, Zeekoevlei (ZA); Dominic Nicholas, Stellenbosch (ZA); Ethan Wade Hunter, Stellenbosch (ZA); Leon Milner Dicks, Stellenbosch (ZA); Nicholas George Enslin, Bo Kaap (ZA); Shaun Wayne Peters, Zeekoevlei (ZA)

(73) Assignee: STELLENBOSCH UNIVERSITY, Stellenbosch (ZA)

(21) Appl. No.: 18/011,876

(22) PCT Filed: Jun. 22, 2021
(86) PCT No.: PCT/IB2021/055503

§ 371 Date: Dec. 21, 2022

(30) Foreign Application Priority Data
Jun. 22, 2020 (GB) 2009513.9

(51) Int. Cl. C12N 9/96; C12N 11/14; C12M 1/40; C12M 1/00; C12N 9/42

(52) U.S. Cl. CPC C12N 9/96; C12N 11/14; C12M 21/18; C12M 29/00; C12N 9/2445; B82Y 5/00

(57) ABSTRACT

The present invention relates to a bioreactor for the catalytic conversion of a substrate to a product using an immobilized enzyme. The immobilized enzyme is a histidine tagged enzyme, which binds to a nickel-nanoparticle coated cellulose matrix which is housed within the bioreactor. The invention also relates to methods of producing products by enzymatic catalysis using the bioreactor of the invention.

### BACKGROUND OF THE INVENTION

Free enzymes in solution react with substrates to produce secondary products. In industrial processes the use of enzymes in this manner is wasteful, as most enzymes are not stable, and cannot be recovered for reuse. Furthermore, the activity of free enzymes in solution are limited by the ability of the enzymes to diffuse to all of the substrate within a given solution, thus reducing their effectiveness in high-throughput processes.

The immobilization of enzymes or whole cells relies on confining and/or anchoring enzymes or cells in or on an inert support structure. This ensures stability and functional reuse of the enzymes. By employing this technique, the immobilized enzymes may be used in a more efficient and cost-effective manner, particularly for industrial use. If done effectively, immobilized enzymes can retain their structural conformation necessary for catalysis.

Immobilized enzymes are generally preferred over immobilized whole cells due their ability to produce secondary products in pure form. However, there are several advantages of using immobilized multi-enzyme systems such as whole cells and/or organelles over immobilized enzymes. Immobilized cells possess natural cofactors and the ability to produce more of a particular enzyme, further, whole cells are particularly suitable for multiple enzymatic reactions. Immobilized multi-enzyme cascade systems have been developed in academia and hold the potential to be transferred over to industry.

There are several advantages of immobilized enzymes, including: (i) immobilized enzymes are stable and more efficient in function, (ii) immobilized enzymes can be reused, (iii) the secondary products produced by the catalytic reaction are enzyme free, (iv) immobilized enzymes may be used in multi-enzyme reaction systems, (v) the control of enzyme function is simple and cheap, (vi) immobilized enzymes are suitable for industrial and medical use, and (vii) immobilized enzymes reduce effluent disposal problems.

There are however, certain disadvantages also associated with the use of immobilized enzymes, such as: (i) the possibility of loss of biological activity of an enzyme during immobilization or while it is in use, and (ii) immobilization is expensive and often requires sophisticated equipment.

There are numerous methods in the art by which proteins, and specifically, enzymes can be immobilized. Each technique has its advantages and disadvantages. The different techniques include adsorption, entrapment, inclusion in microparticles, covalent binding between the enzyme and a support structure (including cyanogen bromide activation, diazotation, peptide bond formation, activation by bi- or poly-functional reagents), cross-linking and coordinated complexes.

Certain enzymes cannot be immobilized, and they have to be used in soluble form e.g. enzymes used in liquid detergents, some diagnostic reagents and food additives. Such enzymes can be stabilized by using certain additives or by chemical modification. Stabilized enzymes tend have longer half-lives, although they cannot be reused. Typical methods of enzyme stabilization used in the art include: solvent stabilisation, substrate stabilisation, stabilisation, stabilisation using polymers, stabilization using salts, stabilisation by chemical modification, stabilisation by rebuilding and stabilisation by site-directed mutagenesis.

More recent technologies allow these solubility dependent enzymes to be immobilized on nanoparticle substrates that do not hinder their function and still allow them to be recaptured and recycled. However, the economic viability of immobilizing these enzymes may negate the need for them to be reused in the first place.

Enzyme immobilization is frequently associated with changes in the properties of the enzyme, particularly kinetic properties. Such changes may include a decrease in enzyme specificity for its substrate. Which may be due to conformational changes that occur when the enzyme gets immobilized. Further, the kinetic constants Kₘ and Vₘₐₓ of an immobilized enzyme could differ from that of the native enzyme. This is as a result of immobilization causing conformational changes in the enzyme, which affects the affinity between the enzyme and its specific substrate.

Immobilized individual enzymes can be successfully used for single-step reactions. They are, however, less suitable for multi-enzyme reactions and for reactions requiring cofactors. For this purpose, whole cells or cellular organelles can be immobilized to serve as multi-enzyme systems. In addition, immobilized cells are sometimes preferred to immobilized enzymes for single reactions, due to cost. For enzymes which depend on the special arrangement of the membrane, cell immobilization is preferred.

Immobilized cells have been traditionally used for the treatment of sewage. The techniques employed for immobilization of cells are almost the same as those used for immobilization of enzymes, with appropriate modifications. Entrapment and surface attachment techniques are commonly used. Gels, and to some extent membranes, are also employed.

Whole cell immobilization is a system in which non-specific enzyme activity is permissible and lower overall efficiency of reactions is also acceptable. This is due to the fact that the target substrate may be metabolized by the cell and the target product not released or released at a lower efficiency. Furthermore, non-specific reaction products may also be released into the effluent and be collected with the final desired product.

Viable cells can be preserved by mild immobilization. Such immobilized cells are particularly useful for fermentations. Sometimes mammalian cell cultures are made to function as immobilized viable cells.

In certain instances, immobilized non-viable cells are preferred over the enzymes or viable cells. This is mainly due to the cost of isolation and purification of specific enzymes. The best example is the immobilization of cells containing glucose isomerase for the industrial production of high fructose syrup.

Both prokaryotic cells and eukaryotic cells may be used in immobilization methods. However, due to the presence of cellular organelles, the metabolism of eukaryotic cells is slow. Thus, for the industrial production of biochemical products, prokaryotic cells are preferred. However, for the production of complex proteins (e.g. immunoglobulins) and for proteins that undergo post-translational modification, eukaryotic cells may be used.

In instances where immobilized enzymes are used in industrial processes, they require an efficient method by which they can be integrated into the process in order to maximize their effectiveness. There are broadly two types of integration methods, namely batch reactors and continuous reactors.

In batch reactors, the immobilized enzymes are incubated together with their specific substrate and the reaction is allowed to take place under constant stirring. As the reaction is completed, the product is separated from the enzyme (usually by denaturation).

Soluble enzymes are commonly used in batch reactors. This presents the problem of separating the soluble enzymes from the products. Further, there is a limitation on the reuse of soluble enzymes. However, special techniques have been developed for the recovery of soluble enzymes, although in many instances these result in loss of enzyme activity.

Stirred tank reactors are the simplest form of batch reactor. They are composed of a reactor fitted with a stirrer that allows good mixing, and appropriate temperature and pH control. However, stirred tank reactors may result in a loss of some enzyme activity. A modification of the stirred tank reactor is a basket reactor. In this system, the enzyme is retained over the impeller blades. Both stirred tank reactors and basket reactors have a well-mixed flow pattern.

Plug flow type reactors are an alternative to flow pattern type of reactors. In these reactors the flow rate of fluids is controlled by a plug system. These plug flow type reactors may be in the form of packed bed or fluidized bed reactor. These reactors are particularly useful when inadequate product formation occurs in flow type reactors. Further, plug flow reactors are also useful for obtaining kinetic data on the reaction systems.

Continuous reactors are reactors in which the substrate is added continuously while the product is removed simultaneously. Immobilized enzymes can be used for continuous operation. Continuous reactors have certain advantages over batch reactors. These include control over the product formation, convenient operation of the system and easy automation of the entire process. There are mainly two types of continuous reactors, namely continuous stirred tank reactors (CSTR) and plug reactors (PR).

Membrane reactors are reactors that are comprised of several membranes with a variety of chemical compositions. The commonly used membrane materials include polysulfone, polyamide and cellulose acetate. The biocatalysts (enzymes or cells) are normally retained on the membranes of the reactor. The substrate is introduced into the reactor while the product passes out of the reactor. Efficient mixing in the reactor can be achieved by using a stirrer. In a continuous membrane reactor, the biocatalysts are held over membrane layers on to which substrate molecules are passed.

In a recycle model membrane reactor, the contents (i.e. the solution containing enzymes, cofactors, and substrates) along with the secondary product are recycled using a pump. The product passes out of the reactor and can be recovered.

There are currently no commercially viable modular systems for immobilization of proteins for use in in vitro biosynthetic reactions, whether that be for laboratory use or commercial use. Current available systems are exorbitantly expensive, the immobilization substrate is dense and not compatible with substrate media found in industry, and many of the immobilization technologies rely on the interaction of active groups on the surface of the peptide with the immobilizing substrate.

### SUMMARY OF THE INVENTION

The present invention relates to a bioreactor for the catalytic conversion of a substrate to a product using an immobilized enzyme. The immobilized enzyme is a histidine tagged enzyme, which binds to a nickel-nanoparticle coated cellulose matrix which is housed within the bioreactor. The invention also relates to methods of producing products by enzymatic catalysis using the bioreactor of the invention.

According to a first aspect of the invention there is provided for an enzyme-polymer conjugate, comprising (i) a histidine tagged enzyme, and (ii) a cellulose matrix, wherein the cellulose matrix is coated with a nickel-nanoparticle, wherein the nickel-nanoparticle is prepared by combining a NaBH₄ solution and a NiCl solution, and wherein the histidine tagged enzyme is immobilized on the cellulose matrix through the formation of a coordinate covalent bond between the histidine tag and the nickel-nanoparticle.

In a first embodiment of the invention the enzyme is selected from the group consisting of α-acetolactate, α-arabinosidase , α-galactosidase, a-rhamnosidase, β-galactosidase, β-glucanase, β-glucosidase, β-glucanase, β-mannanase, γ-lactamase, acetolactate decarboxylase, activase, adenosine deaminase, aminoacylase, aminopeptidase, amylase, amyloglucosidase, asparginase, aspartase, bromelain, carbonic anhydrase, catalase, cellulase, chitinase, chymosin, collagenase, cyclodextrinase, deoxyribonuclease I, dextranase, epimerase, esterase, formate dehydrogenase, galactinol synthase, glucanotransferase, glucoamylase, glucose isomerase, glucose oxidase, glutenase, hemicellulase, hexose oxidase, inulinase, invertase, laccase, lactase, lactate dehydrogenase, leucine dehydrogenase, levanase, lipase, lipoxygenase, lysozyme, methane monooxygenase, monoamine oxidase, muramidase, naphthalene dioxygenases, naphthalene monooxygenase, naringinase, nattokinase, nitrile hydratase, papain, pectinase, pectinesterase, penicillin G acylase, pentosanase, phenoloxidases, phenylalanine dehydrogenase, phytases, polyethylesterase, polygalacturonase, protease, protopectinase, pullulanase, raffinose synthase, rennet, sacrosidase, serratiopeptidase, sphingosine kinase, stachyose synthase, tannase, taxolase, thermolysin, transaminase, transaminase, transglutimases, trypsin, urease, xylanase, and xylose isomerase.

In a second embodiment of the invention the cellulose matrix is cotton. Preferably the cellulose matrix is cotton wool.

In a further embodiment of the invention the histidine tag is a 4x, 5x, 6x, 7x, 8x, 9x, 10x, 11x, 12x, 13x, 14x or 15x histidine tag. In a preferred embodiment, the histidine tag is a 6x or 10x histidine tag.

In one embodiment the histidine tag is fused to the N-terminal of the enzyme. In an alternative embodiment of the invention the histidine tag is fused to the C-terminal of the enzyme.

In a preferred embodiment of the invention the enzyme catalytically converts a substrate into a product. Preferably, the immobilized enzyme has enhanced catalytic activity relative to an enzyme in free solution.

In yet another embodiment of the invention nickel nanoparticle is prepared by combining the NaBH₄ and NiCl. Preferably, the NaBH₄ is present in a solution comprising 10 mM NaBH₄ in 0.2% w/v NaOH and the NiCl is present in a solution comprising 5 mM NiCl.

In a second aspect of the invention there is provided for a method of converting a substrate to a product, the method comprising contacting the substrate with the enzyme-polymer conjugate as described herein.

In a third aspect of the invention there is provided for a bioreactor comprising (i) a fluid distribution chamber, having an inlet and an outlet, and (ii) an enzyme-polymer conjugate, as described herein, contained within the fluid distribution chamber, wherein the histidine tagged enzyme is immobilized on the nickel nanoparticle cellulose matrix through the formation of a coordinate covalent bond between the histidine tag and a nickel nanoparticle bound to the cellulose matrix.

In one embodiment of this aspect of the invention a fluid containing a substrate is passed into the fluid distribution chamber through the inlet. It will be appreciated that the substrate is converted to a product by the immobilised enzyme.

In yet a further embodiment the product is recovered from the fluid distribution chamber via or from the outlet.

In a preferred embodiment of the invention the fluid may contain a cofactor which enhances the activity of the histidine tagged enzyme.

It will be appreciated by those of skill in the art that the cofactor may be either an organic or an inorganic compound.

In a fourth aspect of the invention there is provided for a method for producing a product by enzyme catalysis, the method comprising the steps of (i) introducing a fluid containing a substrate to a bioreactor comprising a fluid distribution chamber, wherein the fluid distribution chamber includes a conjugate, as herein described. Wherein the substrate is converted to a product by means of enzyme catalysis after coming into contact with the immobilized histidine tagged enzyme; and (ii) recovering the product from the bioreactor.

In one embodiment of this aspect of the invention the product is recovered from the fluid distribution chamber via or from an outlet.

In a preferred embodiment of the invention the fluid may contain a cofactor which enhances the activity of the histidine tagged enzyme.

It will be appreciated by those of skill in the art that the cofactor may be either an organic or an inorganic compound.

In a fifth aspect of the invention there is provided for a method for manufacturing a nickel nanoparticle with a simple process, the process comprising or consisting of mixing an NaBH4 solution and a NiCl solution, cooling and stirring the solution and producing nickel nanoparticles.

In one embodiment of the invention nickel nanoparticle is prepared by combining the NaBH₄ and NiCl. Preferably, the NaBH₄ is present in a solution comprising 10 mM NaBH₄ in 0.2% w/v NaOH and the NiCl is present in a solution comprising 5 mM NiCl.

### BRIEF DESCRIPTION OF THE FIGURES

Non-limiting embodiments of the invention will now be described by way of example only and with reference to the following figures:

FIG. 1: Representation of the present bioreactor. The substrate for the reaction is transported through the flow cell via a peristaltic pump, the immobilized enzyme then catalyses the formation of the product which then leaves the flow cell. This reaction is cyclic and the substrate-product media is continuously passed through the enzyme matrix until all available substrate is consumed.

FIG. 2: Microscope image of the nanocoated cellulose substrate that makes up the porous 3D matrix in which the enzymes are immobilized and the biochemical reaction takes place.

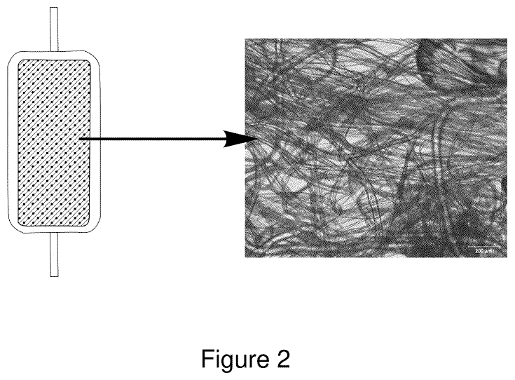

FIG. 3: Representation of the oriented enzyme immobilization strategy as described herein.

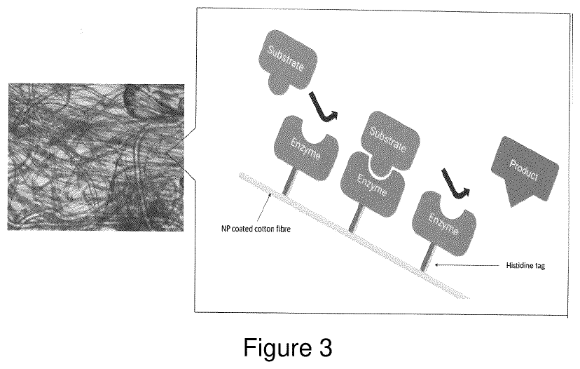

FIG. 4: Representation of the enzyme immobilization procedure compared to commercial competitors.

FIG. 5: Green fluorescent protein immobilized onto the surface of the NP coated cotton wool. This shows that a histidine-tagged protein can be immobilized to the surface of the cotton-NP matrix and remain in its active conformational shape.

FIG. 6: The hydrolysis of pNP-B-glucopyranoside by immobilized beta-glucosidase releases the compound pNP, this changes the colouration of the media from clear to yellow. This experiment demonstrates that enzymes can be immobilized to the matrix and perform their biochemical reactions, thereby remaining active after immobilization.

FIG. 7: The hydrolysis of pNP can be colorimetrically measured using a spectrometer.

FIG. 8: Effect of electrostatic and hydrophobic immobilization techniques on the conformational structure of an enzyme. The change in native protein structure significantly reduces the enzymatic efficiency of the protein.

FIG. 9: An example of a cascade enzyme reaction using three different enzymes: galactinol synthase, raffinose synthase and stachyose synthase. The precursor for the reaction is UDP-galactose which eventually Is synthesized into stachyose through the stepwise reaction.

FIG. 10: Nucleic acid sequence encoding green fluorescent protein (SEQ ID NO:1).

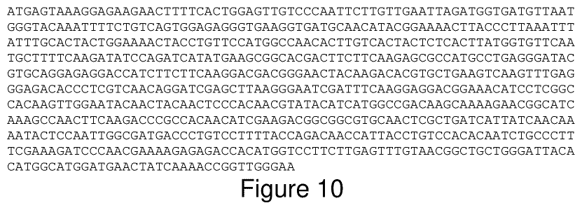

FIG. 11: Amino acid sequence of the green fluorescent protein (SEQ ID NO:2).

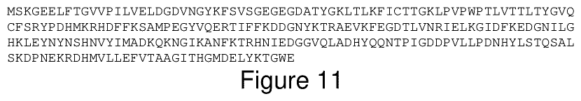

FIG. 12: Nucleic acid sequence encoding the β-glucosidase protein (SEQ ID NO:3).

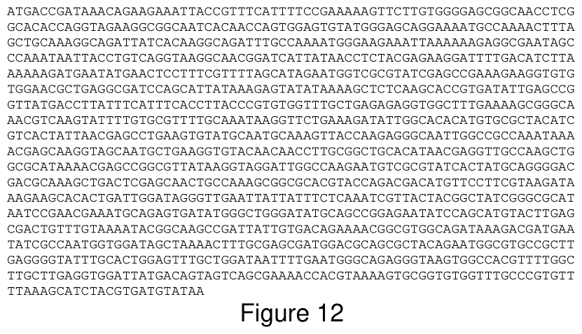

FIG. 13: Amino acid sequence of the β-glucosidase protein (SEQ ID NO:4).

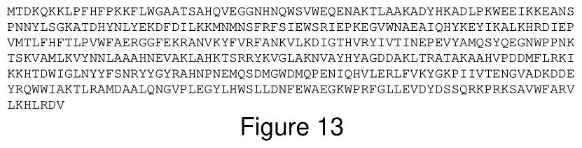

FIG. 14: Nucleic acid sequence encoding the galactinol synthase protein (SEQ ID NO:5).

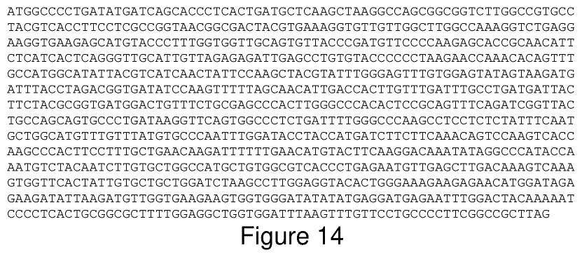

FIG. 15: Amino acid sequence of the galactinol synthase protein (SEQ ID NO:6).

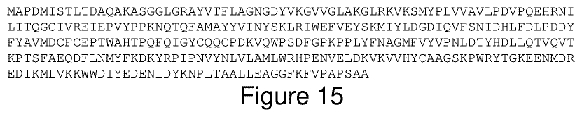

FIG. 16: Nucleic acid sequence encoding the raffinose synthase protein (SEQ ID NO:7).

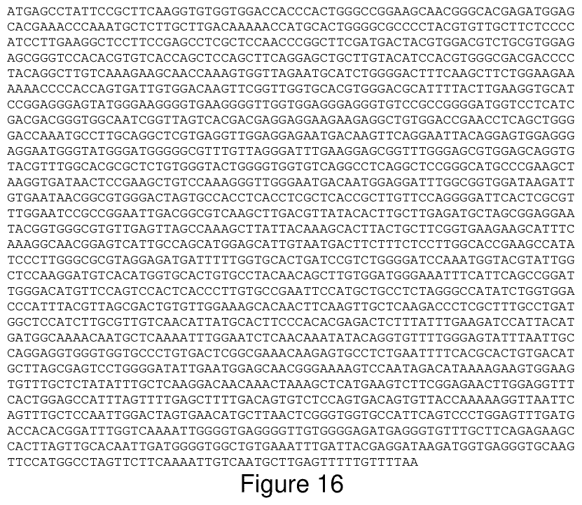

FIG. 17: Amino acid sequence of the raffinose synthase protein (SEQ ID NO:8).

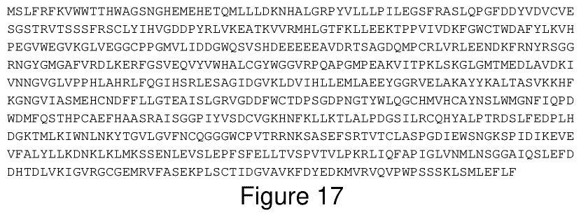

FIG. 18: Nucleic acid sequence encoding the dextranase protein (SEQ ID NO:9).

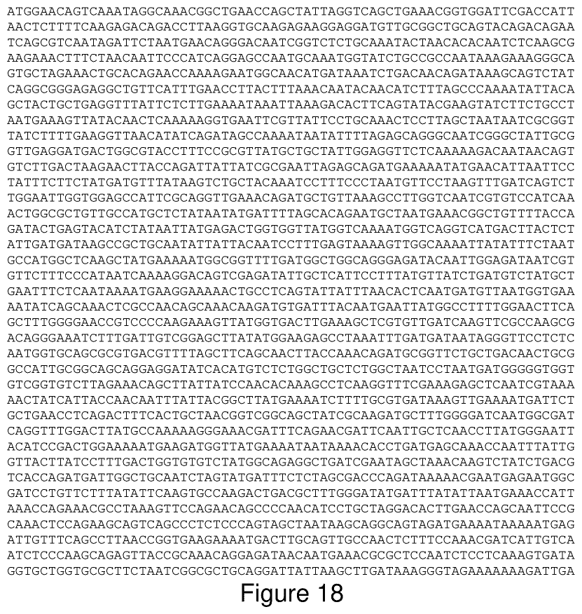

FIG. 19: Amino acid sequence of the dextranase protein (SEQ ID NO:10).

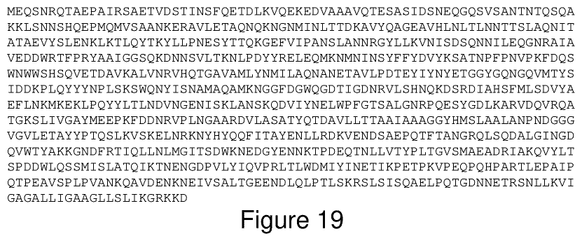

FIG. 20: Nucleic acid sequence encoding the laccase protein (SEQ ID NO:11).

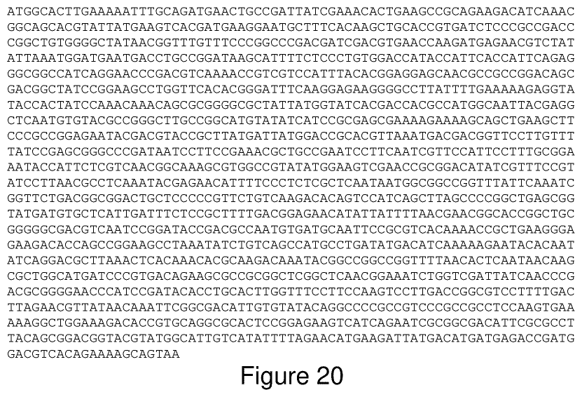

FIG. 21: Amino acid sequence of the laccase protein (SEQ ID NO:12).

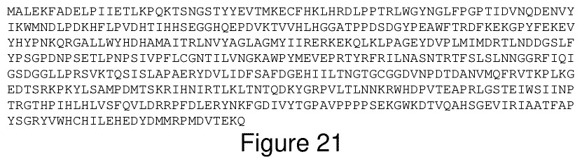

FIG. 22: Nucleic acid sequence encoding the carbonic anhydrase protein (SEQ ID NO:13).

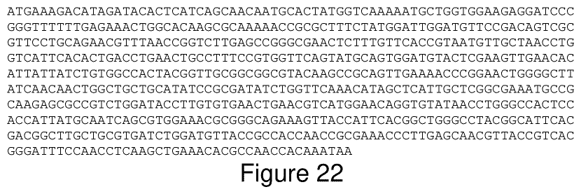

FIG. 23: Amino acid sequence of the carbonic anhydrase protein (SEQ ID NO:14).

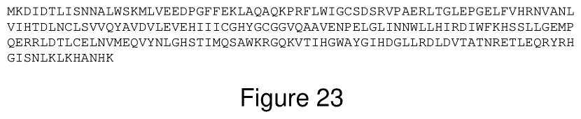

FIG. 24: Nucleic acid sequence encoding the catalase protein (SEQ ID NO:15).

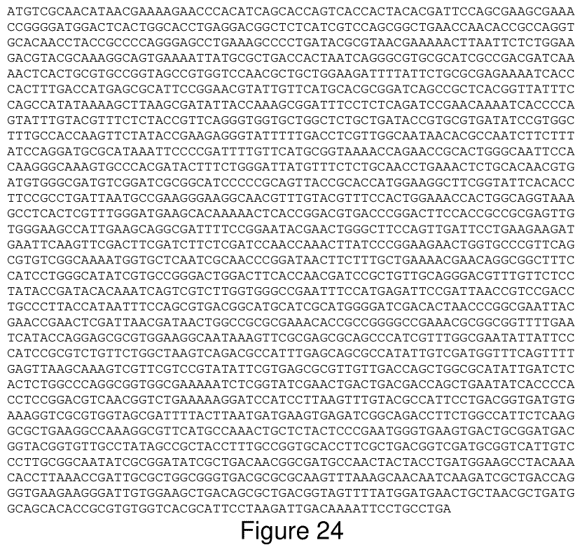

FIG. 25: Amino acid sequence of the catalase protein (SEQ ID NO:16).

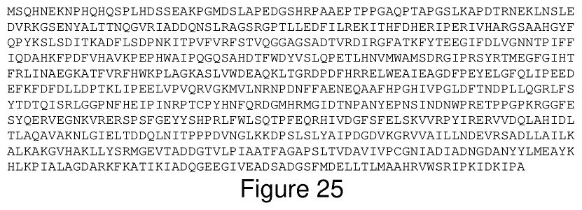

FIG. 26: Nucleic acid sequence encoding the lysozyme protein (SEQ ID NO:17).

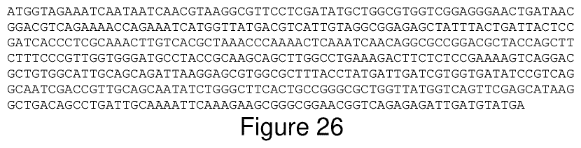

FIG. 27: Amino acid sequence of the lysozyme protein (SEQ ID NO:18).

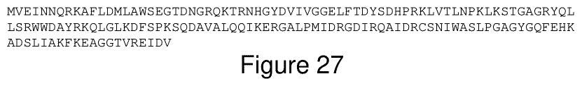

FIG. 28: Standardized nickel nanoparticle synthesis. Standard curve measured at a wavelength of 550 nm for the absorption of nickel nanoparticles. Nickel nanoparticles were synthesized using standard concentrations of NaBH₄ (10 mM, 20 mM, and 40 mM) and respective standard concentrations of NiCl (5 mM, 10 mM, and 20 mM).

FIG. 29: Degrees of polymerization of nickel particles. Field emission scanning electron microscopy (FE-SEM) images of the (A) 10 mM, (B) 20 mM, and (C) 40 mM NaBH₄ samples of undispersed nickel nanoparticle colloids. The observed degree of polymerization is directly proportional to the concentration of substrates (NaBH₄ and NiCl) in the reduction reaction. Images were prepared at CAF, Stellenbosch University, using a Zeiss Merlin™ FE-SEM. Scale is set to represent a length of 500 nm. Brightness=52%. Contrast=28%.

FIG. 30: Dispersed nickel nanoparticles. Field emission scanning electron microscopy (FE-SEM) images of dispersed nickel nanoparticles from the 10 mM NaBH₄ colloid sample. Nanoparticles were resuspended in ethanol with 10% ethylene glycol, and dispersed using an ultrasonic water bath. (A) Independent nickel particle of approximately 300-500 nm in diameter. (B) Dispersed nickel nanoparticles of approximately 150 nm in diameter. Images were prepared at CAF, Stellenbosch University, using a Zeiss Merlin™ FE-SEM. Scale is set to represent a length of 300 nm. Brightness=52%. Contrast=28%.

FIG. 31: GFP immobilization to nickel nanoparticles. (A) Bright field microscopic image of nickel nanoparticles of the 10 mM colloid. (B) Fluorescent light microscopic image with GFP filter of corresponding nickel nanoparticles presenting immobilized GFP. Images were captured by the Zeiss Axiocam at 40× magnification and analysed using the Zen 2 computer software.

FIG. 32: Extended protein stability on nickel nanoparticles. Matching samples as taken in FIG. 31 were stored at room temperature for two weeks, with subsequent microscopic analysis. (A) Bright field microscopic image of a nickel nanoparticle of the 10 mM colloid. (B) Fluorescent light microscopic image with GFP filter of the corresponding nickel nanoparticle presenting immobilized GFP. Images were captured by the Zeiss Axiocam at 40X magnification and digitally analysed using the Zen 2 computer software.

### SEQUENCE LISTING

The nucleic acid and amino acid sequences listed in the accompanying sequence listing are shown using standard letter abbreviations for nucleotide bases, and the standard three letter abbreviations for amino acids. It will be understood by those of skill in the art that only one strand of each nucleic acid sequence is shown, but that the complementary strand is included by any reference to the displayed strand. In the accompanying sequence listing:

SEQ ID NO:1—nucleic acid sequence encoding green fluorescent protein.

SEQ ID NO:2—amino acid sequence of the green fluorescent protein.

SEQ ID NO:3—nucleic acid sequence encoding the β-glucosidase protein.

SEQ ID NO:4—amino acid sequence of the β-glucosidase protein.

SEQ ID NO:5—nucleic acid sequence encoding the galactinol synthase protein.

SEQ ID NO:6—amino acid sequence of the galactinol synthase protein.

SEQ ID NO:7—nucleic acid sequence encoding the raffinose synthase protein.

SEQ ID NO:8—amino acid sequence of the raffinose synthase protein.

SEQ ID NO:9—nucleic acid sequence encoding the dextranase protein.

SEQ ID NO:10—amino acid sequence of the dextranase protein.

SEQ ID NO:11—nucleic acid sequence encoding the laccase protein.

SEQ ID NO:12—amino acid sequence of the laccase protein.

SEQ ID NO:13—nucleic acid sequence encoding the carbonic anhydrase protein.

SEQ ID NO:14—amino acid sequence of the carbonic anhydrase protein.

SEQ ID NO:15—nucleic acid sequence encoding the catalase protein.

SEQ ID NO:16—amino acid sequence of the catalase protein.

SEQ ID NO:17—nucleic acid sequence encoding the lysozyme protein.

SEQ ID NO:18—amino acid sequence of the lysozyme protein.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

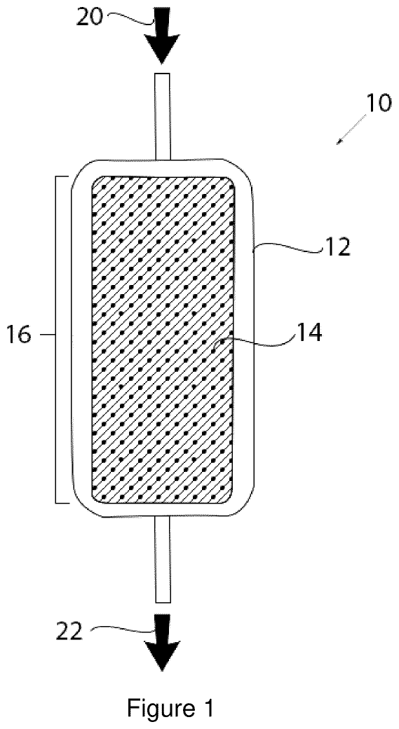

According to a preferred embodiment, as illustrated in FIG. 1, a bioreactor (10) comprises a fluid distribution chamber (12), having an inlet (20) and an outlet (22). The fluid distribution chamber (12) is filled with a nickel nanoparticle coated cellulose matrix (14) comprising a histidine tagged fusion protein. The histidine tagged fusion protein is immobilized on the nickel nanoparticle coated cellulose matrix (14) through the formation of a coordinate covalent bond between the histidine tag and the nickel nanoparticles which are bound to the cellulose matrix (14).

A fluid containing a substrate is passed into the fluid distribution chamber (12) through the inlet (20). The substrate is converted to a product by the fusion protein through enzymatic catalysis of the substrate by the fusion protein. It will be appreciated that the fusion protein consists of a histidine tag fused to an enzyme. The enzyme catalyses the reaction to convert the substrate to the product. The histidine tag may be a 4x, 5x, 6x, 7x, 8x, 9x, 10x, 11x, 12x, 13x, 14x or 15x histidine tag. Preferably, the histidine tag is a 6x or 10x histidine tag.

The enzyme may be any enzyme used in an industrial processes and includes, but is not limited to and enzyme selected from the group consisting of α-acetolactate, α-arabinosidase, α-galactosidase, a-rhamnosidase, β-galactosidase, β-glucanase, β-glucosidase, β-glucanases, β-mannanase, γ-lactamase, acetolactate decarboxylase, activase, adenosine deaminase, aminoacylase, aminopeptidase, amylases, amyloglucosidase, asparginase, aspartase, bromelain, carbonic anhydrase, catalase, cellulase, chitinase, chymosin, collagenase, cyclodextrinase, deoxyribonuclease I, dextranase, epimerase, esterases, formate dehydrogenase, galactinol synthase, glucanotransferase, glucoamylase, glucose isomerase, glucose oxidase, glutenases, hemicellulases, hexose oxidase, inulinase, invertase, laccase, lactase, lactate dehydrogenase, leucine dehydrogenase, levanase, lipase, lipoxygenase, methane monooxygenase, monoamine oxidase, muramidase, naphthalene dioxygenases, naphthalene monooxygenase, naringinase, nattokinase, nitrile hydratase, papain, pectinase, pectinesterase, penicillin G acylase, pentosanase, phenoloxidases, phenylalanine dehydrogenase, phytases, polyethylesterase, polygalacturonase, proteases, protopectinase, pullulanase, raffinose synthase, rennet, sacrosidase, serratiopeptidase, sphingosine kinase, stachyose synthase, tannase, taxolase, thermolysin, transaminase, transaminase, transglutimases, trypsin, urease, xylanase, xylose isomerase.

Most preferably, the enzyme is selected from the group consisting of green fluorescent protein (SEQ ID NO:2), β-glucosidase (SEQ ID NO:4), galactinol synthase (SEQ ID NO:6), raffinose synthase (SEQ ID NO:8), dextranase (SEQ ID NO:10), laccase (SEQ ID NO:12), carbonic anhydrase (SEQ ID NO:14), catalase (SEQ ID NO:16) and lysozyme (SEQ ID NO:18). It will be appreciated that once cloned into the relevant vector, the expressed protein will contain a histidine tag. It will further be appreciated by those of skill in the art that any histidine-tagged enzyme could be used as the enzyme in the enzyme polymer conjugate.

The product that is produced by the enzymatic reaction between the substrate and the immobilized enzyme can be harvested from the fluid distribution chamber via the outlet (22).

The cellulose matrix (14) is preferably a cotton matrix, most preferably the cotton matrix is cotton wool.

The histidine tag of the fusion protein may either be included on the N-or the C-terminal of the protein.

It will be appreciated that cofactors that are required to improve enzyme catalysis may be introduced into the fluid distribution chamber (12) in the fluid. It will further be appreciated that the cofactor may be an organic or inorganic cofactor.

The present invention relates to a bioreactor comprising functionally immobilized enzymes on a three-dimensional, microporous cellulose matrix for use in the in vitro organic synthesis of commodity metabolites (antibiotics, antidepressants, food additives, etc), as a treatment device in the beverage and wine industry, and a device to aid in water purification.

The device consists of four main components (FIG. 1). These segments are the modular flow cell, porous cellulose matrix, nanoparticle capture agent and the immobilized enzyme. The flow cell has two major functions, it serves to house all other components in a compact form and allows for the flow of substrate through the enzymatically active matrix, thus serving as a self-contained reaction vessel. Additionally, due to its modular design it can be arranged in series with other flow cell modules to couple multienzyme reactions together, while keeping the proteins separated. Thus, allowing different reaction conditions to be set up within a single reaction system (i.e. temperature, flow rate, pH, substrates).

The second component is the porous cellulose matrix. This component is housed within the flow cell and is the solid, three dimensional support which the enzymes will be immobilized on and the substrate media passed through. It is therefore the site in which the biochemical reactions will occur. It is solely composed of a uniform mass of simple commercial cotton wool that is treated with the nanoparticle capture agent. Cotton wool is used as an immobilization matrix as it is an abundant and low-cost material. Additionally, cotton wool is inert and stable across a range of conditions.

The third component is the nanoparticle capture agent. Nickel has traditionally been used for the purification of proteins due to its affinity and binding to histidine residues contained within proteins. Nickel-histidine binding has been utilized for decades in commercially available nickel-nitrilotriacetic acid (Ni-NTA) agarose columns. Ni-NTA columns are used for the purification of proteins from expression media in a laboratory setting. They are not suitable for use as a platform for in vitro organic synthesis of compounds or coupled to industrial processes. This is due to the fact that the columns tend to be very expensive. Further agarose is generally used as the matrix in these columns and the density of the agarose makes it difficult for complex substrate media to be passed through it. In the present invention the inventors have adapted the affinity that nickel has for histidine in a novel approach.

Nickel nanoparticles are produced via a novel synthesis method developed for the purpose of enzyme immobilization on the cellulose substrate. Briefly, NiCl₂ is reduced using NaBH₄ under heat in a non-aqueous environment to yield Ni° nanoparticles (hereinafter NPs). The NPs have the ability to bind to histidine residues present in the proteins. Additionally, the NPs have certain attributes that NiCl₂ does not possess, which makes it preferable for use in a modular reactor. The inventors have found that the NPs are capable of permanently coating the cellulose matrix, thus conferring the ability to bind histidine to the cotton wool and allowing the catalytic proteins to be immobilized on the cellulose matrix. Additionally, the NPs are paramagnetic and can be collected from the solution using a simple neodymium magnet. The most significant advantage of this strategy is that the system can only immobilize specific desirable proteins that contain an engineered affinity tag.

The benefits of using the novel method of nickel nanoparticle synthesis disclosed herein are (i) the use of a low temperature reaction environment for the production of the nickel nanoparticles, (ii) the lack of a solvent as a reaction substrate, (iii) the lack of a chemical reflux step, (iv) the use of only two primary substrates, and (v) the resulting stability of the synthesized nanoparticles.

The final component is the enzyme catalyst itself. These proteins perform the biochemical reaction of the system and are irreversibly immobilized onto the surface of the cellulose matrix. Nucleic acids encoding the enzymes of interest were ligated into a histidine-tagged vector system. Other enzyme immobilization technologies immobilize enzymes based on functional groups present on the exterior of the enzyme, whereas in the present invention a user can immobilize any enzyme that contains a histidine-tag.

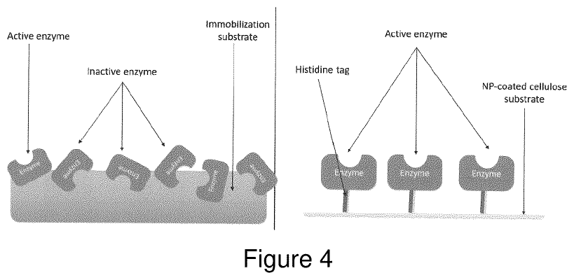

The enzymes used in this process are engineered to contain a histidine tag, preferably a deca-histidine tag (10x-his) on the N or C-terminal end of the proteins. The histidine tag present on the end of the protein is captured by the NP-coated cellulose matrix and the proteins are immobilized to the cellulose platform via their affinity for the NPs. This system is more advantageous than other enzyme immobilization techniques as the active sites of the enzyme are not altered by the immobilization process and are available for conversion of the substrates introduced in the media (FIG. 4).

The present invention is an entirely self-contained bioreactor that can be reused multiple times without a loss of the enzyme, or with a minimal loss of enzyme activity. The novel immobilization technique utilized in the present invention allows for greater exposure of the enzyme active site to the substrate by orienting the enzyme correctly, while simultaneously overcoming the limits of diffusion by bringing the substrate to the enzyme by way of cycling the substrate containing media through the flow cell. Furthermore, the materials used in the construction of the present invention are low cost, “off-the-shelf” components that reduce the cost of the invention by orders of magnitude over that of any competitor. Finally, the modular design of the present invention also allows for rapid scalability from niche laboratory experiments to large scale industrial operations with very little effort.

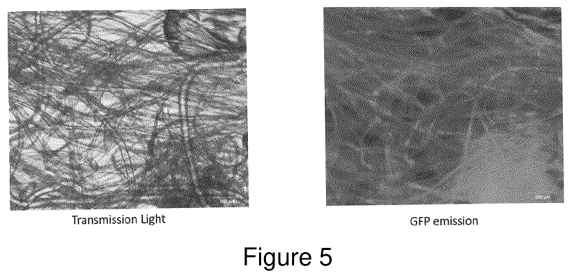

The functionality of the immobilization was confirmed in two experiments, in the first a histidine tagged green fluorescent protein (GFP) was immobilized onto the surface of the cellulose-NP substrate and then viewed using fluorescent microscopy. If the surface of the cellulose-NP substrate emitted green light under the microscope, it can be deduced that the GFP protein was present and immobilized, while not losing the ability to fluoresce thereby maintaining its active conformation (FIG. 5).

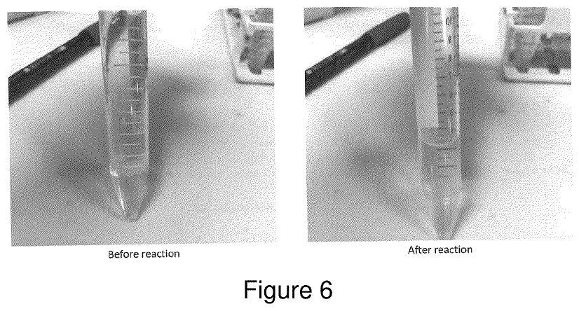

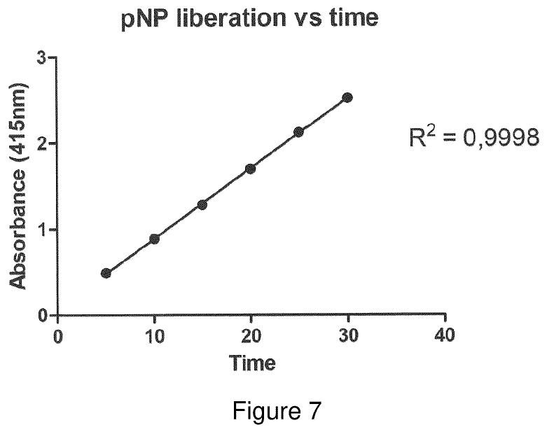

The second experiment was performed to confirm that enzymatically active proteins immobilized onto the cotton-NP matrix did not lose their ability to perform their biochemical reactions. To do this, a N-terminal histidine tagged beta-glucosidase enzyme was immobilized onto the surface of the cotton-NP matrix. The chemical substrate pNP-β-D-glucopyranoside was then cycled through the enzyme immobilized matrix. Functional enzyme immobilization allows for the hydrolysis of the pNP-β-D-glucopyranoside substrate and the release of pNP can be detected visually by a change in the colour of the reaction media from clear to yellow. This can be further quantified using spectroscopy. This experiment was successful and proved that enzymes could be immobilized onto the surface of the substrate and remain biochemically active (FIGS. 6 and 7).

The concept of enzyme immobilization has been thoroughly investigated in recent years and holds major potential in a number of commercial and academic practices. However, cost and efficiency drawbacks currently prevent these technologies from being commercially viable. The present invention overcomes both of these major limitations. The immobilization matrices that current technologies employ are mostly synthetically produced at high cost, often involving complex biochemical processes which require the use of toxic compounds that can reduce the catalytic activity of the enzyme (Table 1). Furthermore, the mechanisms by which these matrices immobilize the proteins often reduce the activity of the enzyme in two distinct manners.

The first is by reducing access of the substrate to the active site of the enzyme by the suboptimal orientation of the enzyme upon immobilization. This is known as steric hindrance of the enzyme active site and results in the loss of enzyme activity, as a result of the key active site being blocked and reactions not being able to take place (FIG. 4). This occurs due to the method of immobilization inherent in other systems. Examples of such systems are the interaction between the activated resin (immobilization matrix) with the primary amines (—NH₂) or carboxyl groups (—COOH), or other target groups (lysine residues, etc), on the surface of the protein of interest (Table 1). The issue is that these groups can be found over the entire surface of the protein. This leads to non-oriented immobilization of the protein, which can result in steric hindrance. Furthermore, these groups are not specific to proteins of interest, therefore requiring expensive purification of the proteins prior to immobilization, to ensure that only the specific protein of interest is immobilized.

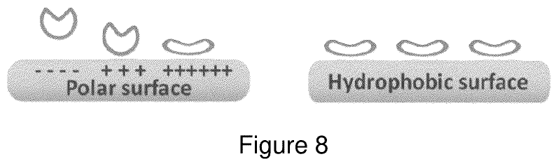

Secondly, the immobilization process of other technologies can result in a negative conformational change in the structure of the enzyme resulting in a further loss of activity. This can be seen in the case of enzymes which are immobilized by utilization of electrostatic or hydrophobic interactions (FIG. 8). Proteins may be misfolded due to their interaction with the immobilizing surface. Because catalytic activity is strongly linked with the structural conformity of the protein, these effects have a negative impact on the reaction rate of the biochemical processes (FIG. 8).

The present overcomes these issues by making use of an organic cellulose matrix, which provides an affordable immobilization complex, as well as limiting any intensive synthetic processes that require toxic chemicals. The cellulose matrix is also extremely permeable compared to the agarose or silica alternatives, subsequently resulting in an increased rate of mass transfer and making it an attractive platform for large volume reactions. Additionally, the immobilization process utilized by the present invention is based on the affinity between the Ni-NPs and the histidine tag engineered on the terminal domains of the proteins of interest. This has a twofold benefit, the first is overcoming the negative effect of steric hindrance, as a result of the histidine tag acting as a directional bridge and spacer between the immobilization platform and the enzyme thus ensuring that the active sites of the enzyme are not blocked from interacting with their substrates. Secondly, the histidine tag ensures that only the protein of interest is immobilized, since this tag is not found in other proteins, which is in contrast to the use of amine or carboxyl groups that are found extensively on other proteins. Finally, because the present invention relies on the gentle interaction of Ni-NP and histidine and not on physical or entrapment immobilization approaches, the conformation of the enzyme is not negatively modified by immobilization. The combination of these features drastically increases enzyme activity, stability and reusability.

TABLE 1
Enzyme immobilization platforms

| | Pierce NHS- | AminoLink | AminoLink | SulfoLink | | |
|---|---|---|---|---|---|---|
| | Activated | Coupling | Plus Coupling | Coupling | GlycoLink | CarboxyLink |
| | Agarose | Resin | Resin | Resin | Coupling | Coupling |
| Target | —NH₂ | —NH₂ | —NH₂ | —SH | —CHO | —COOH |
| Support | 6% agarose | 4% agarose | 4% agarose | 6% agarose | Polyacrylamide/ | 4% agarose |
| | | | | | azlactone | |
| | | | | | copolymer | |
| Coupling | >25 mg/mL | 1-20 mg/mL | 20 mg/mL | 20 mg/mL | 1-10 mg/ml | 5 mg/mL |
| capacity | resin | resin | resin | resin | resin | resin |
| Coupling time | 30 min | 4 hr | 4 hr | 2-3.5 hr | 30 min | 4 hr |
| Recommended | Proteins | Proteins, | Proteins, | Proteins, | Glycoproteins, | Unmodified |
| for | | antibodies | antibodies | peptides, | polyclonal | peptides |
| | | | | antibodies | antibodies | |
| Advantages | High coupling | High coupling | High coupling | Allows for | Correctly | Flexible and |
| | efficiency and | efficiency | efficiency | gentle elution | orients | gentle |
| | capacity | | | | antibodies | coupling |
| | | | | | during coupling | conditions |
| Disadvantages | Narrow pH | May be | May be | Protein must | Not | Not |
| | range (7-9); | coupled at | coupled at | be reduced | recommended | recommended |
| | non-oriented | antigen- | antigen- | first; non- | for monoclonal | for antibodies |
| | protein | binding site, | binding site, | oriented | antibodies | |
| | immobilization | non-oriented | non-oriented | protein | | |
| | | protein | protein | immobilization | | |
| | | immobilization | immobilization | | | |
| | | | Immo | | | |
| | | WO19/ | Bead | PolyLink | | |
| | | 186452 | Kit | Epoxy Kit | Zymotronix | Cell Mosaic |
| | Target | Lysine | Multiple | Covalent | | Cys amino |
| | | Residues | | binding to | | acid covalent |
| | | | | methacrylate | | bond on the |
| | | | | and styrene | | C-or N- |
| | | | | beads | | terminal |
| | Support | Styrene and | Beads | Styrene | Magnetic | 4% agarose |
| | | maelic | | beads | nanoparticles | |
| | | anhydride | | | | |
| | Coupling | 4-10 mg/mL | — | >150 mg/ml | — | — |
| | capacity | | | | | |
| | Coupling time | 1 hr | | 20 hrs | | |
| | Recommended | Proteins | Proteins | Proteins | Proteins | Proteins |
| | for | | | | | |
| | Advantages | — | A variety of | Fairly large | Ease of | Custom kit |
| | | | carriers for | volume | separation | with peptide |
| | | | either | reactions. A | and recovery | modifications |
| | | | covalent, | variety of | of enzymes | for Cys |
| | | | absorbed, | resins | | amino acid |
| | | | cationic or | | | |
| | | | anionic | | | |
| | | | binding | | | |
| | Disadvantages | Slow | Requires in- | Requires in- | One pot | Expense of |
| | | diffusion | depth | depth | reaction | agarose and |
| | | rates | optimisation | optimisation | | reduced flow |
| | | | i.e. time | i.e. time | | rate of |
| | | | | | | substrate |

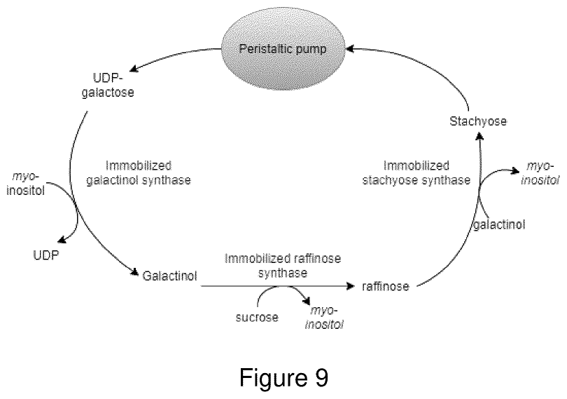

The three-dimensional modular flow cell design allows for substrates to be used in a recyclable manner, offering maximum product throughput. The present invention can therefore accommodate a variety of substrates and cofactors, as well as multi-enzyme networks, all linked in a series of modular cassettes. This ultimately allows for the potential of a fully integrated cell free environment for entire biosynthetic pathways (FIG. 9).

The production of the present enzyme immobilization technology greatly benefits enzyme binding and functionality and additionally limits the presence of toxic compounds during the production of the immobilization platform.

Ultimately, present invention has the potential to be used in order to synthesize a variety of commercially and environmentally favourable products, at low cost. Additionally, this technology holds great potential in a multitude of analytical, diagnostic and industrial applications.

The following examples are offered by way of illustration and not by way of limitation.

### EXAMPLE 1

Nanoparticle (NP) Synthesis

Nickel nanoparticles were synthesized in a 1 L round bottom flask containing 20 mL ethylene glycol (Sigma-Aldrich), 100 mg of nickel chloride (Sigma-Aldrich), 500 mg of polyvinylpyrrolidone (Sigma-Aldrich) and 20 μL of deionised water.

The mixture was then introduced to a rotavapor in a 90° C. water bath and mixed at ˜80% RPM under vacuum until the contents were completely transparent. Following this, 200 mg of sodium borohydride (Sigma-Aldrich), acting as a reducing agent, was added to the solution.

The solution began to turn black and was placed on to the rotavapor for 30 minutes with mixing at 90° C. Following this, 50 mg of sodium borohydride and 10 μL of deionised water were added to the solution every 30 minutes until a total of 400 mg of sodium borohydride was added and the solution had been incubating for a total of 2.5 hours.

The solution was then removed from the rotavapor and placed in a glass vial, covered in tinfoil (to prevent photo-oxidation) and stored at 4° C. This solution will be referred to as the “NP stock solution”.

Cotton NP Functionalization

In order to functionalize the cotton support matrix with the nickel nanoparticles a working solution of NPs, containing 100 μL of the NP stock solution was resuspended in 24 900 μL of chloroform to form a stable colloidal solution, was made up.

Following this, a 15 mg piece of cotton wool was placed in 10 mL of working solution and mixed for one minute. After which, the cotton wool was removed and drained of any liquid chloroform and allowed to dry at room temperature. The cotton wool was then placed back into the chloroform and the process was repeated twice more.

This increases the efficiency of association between the NPs and the cotton wool. To remove any NPs that are weakly associated with the cotton wool matrix, the cotton wool is then washed twice in 15 mL of 95% ethanol, followed by a final wash in 15 mL of deionised water.

Protein Production and Expression

The vectors pRSF-Duet1:GFP and psFOX-oxB20:BGL4 were provided by the Dicks laboratory and Dr. Bianke Loedolff, respectively. These vectors contain nucleic acid sequences encoding the green fluorescent protein (GFP) (SEQ ID NO:2) and beta-glucosidase enzymes (BGL4) (SEQ ID NO:2) (the respective nucleic acid sequences are SEQ ID NO:1 and SEQ ID NO:3).

These two proteins were expressed in two distinct processes dictated by their specific vectors. For the GFP, the vector was transformed into the E. coli BL21 (DE3) host via the heat shock transformation method (Addgene). A positive colony that was confirmed by PCR was used to inoculate a 5 mL Luria Broth (LB) culture, supplemented with 50 mg/mL kanamycin and grown overnight at 37° C. with aeration. The following day, the overnight culture was used to inoculate 400 mL of sterile terrific broth supplemented with 50 mg/ml kanamycin. The culture was grown at 37° C. with aeration until an OD₆₀₀ of 0.6 was reached. Following this, 1 mM Thio-B-D-galactopyranoside (IPTG) was added to the culture and it was incubated at room temperature for 24 hours. The cells were centrifuged (8 000 g, 30 min at 4° C.) and the pellet resuspended in 40 mL lysis buffer (10 mM Tris-HCl (pH 8.0), Triton-X100 (1%), lysozyme (1 mg/mL), DNase (1 ng/μL) and RNase (10 ng/μL)). The cell suspension was incubated on ice for 30 min, followed by disruption through sonication on ice (3 times at 70% power output, 50% pulses for 3 min). Lysed samples were centrifuged (12 500 g, 2 hours, 4° C.) and proteins in the supernatant purified by IMAC. The supernatant was adjusted to a final imidazole concentration of 40 mM and loaded onto Ni-Sepharose 6 Fast Flow columns (GE Healthcare, South Africa) which were pre-equilibrated with SB40 (SB buffer containing 40 mM imidazole). Columns were washed with SB40 and His-tagged fusion proteins eluted using SB500 (SB buffer containing 500 mM imidazole).

For BGL4, the vector was transformed into the E. coli BL21 (DE3) host via the heat shock transformation method (Addgene). A positive colony that was confirmed by PCR was used to inoculate a 5 mL Luria Broth (LB) culture, supplemented with 50 mg/mL kanamycin and grown overnight at 37° C. with aeration. The following day, the overnight culture was used to inoculate 400 mL of sterile terrific broth supplemented with 50 mg/ml kanamycin. The culture was grown at 37° C. with aeration until an OD₆₀₀ of 0.6 was reached. Following this, the culture was incubated at room temperature for 24 hours.

The cells were then centrifuged (8000 g, 30 min at 4° C.) and the pellet resuspended in 40 mL lysis buffer (50 mM HEPES (pH 7.5), Triton-X100 (1%), lysozyme (1 mg/mL), DNase (1 ng/μL), RNase (10 ng/μL), MgCl₂ (5 mM), EDTA (1 mM), Na-ascorbate (50 mM), benzamidine (1 mM) and PMSF (1 mM)). The cell suspension was incubated on ice for 30 min, followed by disruption through sonication on ice (3 times at 70% power output, 50% pulses for 3 min). Lysed samples were centrifuged (12 500 g, 2 hours, 4° C.) and proteins in the supernatant purified by IMAC. The supernatant was adjusted to a final imidazole concentration of 25 mM and loaded onto Ni-Sepharose 6 Fast Flow columns (GE Healthcare, South Africa) which were pre-equilibrated with EQ buffer (HEPES (50 mM), MnCl₂ (2 mM)). Columns were then washed with EQ25 (EQ buffer and imidazole (25 mM)) and His-tagged fusion proteins eluted using EQ500 (EQ buffer and imidazole (500 mM)).

Protein Immobilization

The immobilization of N-terminal histidine tagged (6X) GFP and N-terminal tagged (10X) BGL4 (beta-glucosidase) were performed identically. A 1/20 working solution of each peptide was first made up by diluting 500 μL of purified protein in 19 500 μL of working buffer consisting of either Tris-HCl (10 mM, pH 8.0) or HEPES (50 mM, pH 7.5) for GFP and BGL4, respectively. To immobilize the peptides onto the functionalized cotton wool matrix, a peristaltic pump was used to transfer the peptide working solution cyclically through the cotton matrix which was housed within a modified syringe. The functionalized cotton wool contained within the modified syringe served as a “bioreactor” and here acts as a prototype of the bioreactor of the present invention. The protein working solutions were passed through the prototype bioreactor for 30 minutes and then washed with 10 mL working buffers of Tris-HCl (10 mM, pH 8.0) or HEPES (50 mM, pH 7.5) for GFP and BGL4, respectively. This washing step removes any protein that has not been immobilized. The protein activated enzyme-polymer conjugates are now ready for a biocatalysis reaction (for BGL4) or fluorescent microscope analysis (for GFP).

Enzymatic Conversion Using the System

In order to demonstrate that the immobilized BGL4 enzyme retained its β-glucosidase enzyme activity, a continuous hydrolysis reaction was performed using the synthetic substrate p-nitrophenyl-β-glucopyranoside (pNPG).

The immobilized enzyme catalysed continuous hydrolysis reaction was performed in a cyclic batch system by cycling 20 mL of reaction solution (50 mM HEPES (pH 7.5), 4 mg/mL of pNPG) through the enzyme-polymer conjugate which contained the immobilized BGL4 peptide. At five minute intervals, 100 μL of the reaction solution was transferred to a 96-well microtiter plate containing 200 μL of sodium carbonate (0.5 M) and measured at 415 nm.

The cleavage of the sugar moieties from the pNPG by the β-glucosidase liberates p-nitrophenol that is quantified spectrophotometrically at a wavelength at or around 415 nm. An increase in absorbance at these wavelengths indicates that the enzyme is catalytically active when immobilized. Furthermore, the liberated p-nitrophenol will shift the colour of the reaction from clear to yellow, a clear indicator of enzyme activity.

As can be seen in FIGS. 6 and 7, the immobilized beta-glucosidase enzyme retained its catalytic activity and was highly efficient at cleaving the p-nitrophenol from the glucosyl sugar moiety.

### EXAMPLE 2

Nickel Nanoparticle Synthesis

Nickel nanoparticles (NPs) were synthesized by a novel method broadly based on the reduction of nickel chloride by sodium borohydride, under highly specific and controlled conditions.

Specifically, a low concentration range of sodium borohydride (NaBH₄, Sigma-Aldrich) was prepared using deionized water (dH₂O) with trace sodium hydroxide, to prevent the spontaneous reduction of NaBH₄ in dH₂O (6 mL, 10 mM; 20 mM; 40 mM NaBH₄ in 0.2% w/v NaOH). Nickel chloride (NiCl, Sigma-Aldrich) was prepared in solution at exactly half the concentration of the respective NaBH₄ (2 mL, 5 mM; 10 mM; 20 mM NiCl, respectively, in dH₂O). Both solutions were cooled on ice prior to the synthesis reaction.

Ensuring agitation by vortex, the ice cold NiCl solution was gradually added to the ice cold NaBH₄ solution. This mixture was continuously vortexed and kept cold on ice in brief intervals throughout vortexing. The reaction proceeded slowly, directly correlating with the concentration of reagents, with the observable reaction occurring when the solution turned grey as nickel NPs were synthesized. Agitation was suspended immediately after the observable reaction occurred, and each sample was placed on ice.

Spectrometry

The standard range of nickel nanoparticle colloids were analyzed by spectrometry for nanoparticle density using a microplate reader (VERSA max, Tunable). Each colloid sample was prepared in a 96-well microtiter plate, with dH₂O as a blank, and absorbance readings were measured at a wavelength of 550 nm (100 μL in each well, A_{550 nm}).

Nickel Nanoparticle Colloid Dispersion and Storage

The nickel NP solution was centrifuged (10 000×g, 2 min) and the supernatant discarded. The remaining nickel NPs were washed with dH₂O and subsequently separated by centrifugation (10 000×g, 2 min) and the supernatant discarded. The nickel NP pellet was resuspended in ethanol and the colloidal solution was subsequently stabilized by the addition of ethylene glycol (10% v/v in colloid). The resulting nickel NP colloid was placed in an ultrasonic water bath and sonicated for 10 min to separate any nickel NP aggregates.

Polymer Characterization by Scanning Electron Microscopy (SEM)

Characterization based on size and shape of the synthesized nickel polymers and nanoparticles were conducted at the Stellenbosch University Central Analytical Facility (CAF, EM Unit). High resolution nano-scale images were produced using a field emission scanning electron microscope (MERLIN™ FE-SEM, Zeiss).

Protein Immobilization to Nickel NPs

Protein immobilization was performed using the N-terminal histidine tagged (6X) GFP. The GFP was isolated as previously described .

The nickel NP stock colloid was centrifuged (10 000×g, 2 min) and the supernatant discarded. The nickel NP pellet was subsequently washed with dH₂O by vortexing, followed by centrifugation (10 000×g, 2 min) and the supernatant discarded. This washing process was repeated three times to ensure all of the solvent and ethylene glycol were removed.

GFP was prepared in a 10× dilution in a phosphate buffered solution (pH 5.0, 1 M). The GFP dilution was added to the washed nickel NP pellet and vortexed. The mixture was subsequently incubated at 37° C. for 1 h. Following incubation, the sample was centrifuged (10 000×g, 2 min) and the supernatant discarded. The remaining nickel NP-GFP pellet was washed with TE buffer solution (pH 8.0, 10 mM Tris, 1 mM EDTA) three times, as previously described. The nickel NP-GFP sample was then analysed using a fluorescent microscope equipped with a GFP filter (Axio Scope.A1, Zeiss), presenting the nickel NP-GFP stable complex.

The samples were kept at room temperature for two weeks before a subsequent analysis using the fluorescent microscope to test for protein stability over time. This stability test presented results coherent with the initial analysis, suggesting a high level of stability for the immobilized GFP.

Standardized Nanoparticle Synthesis

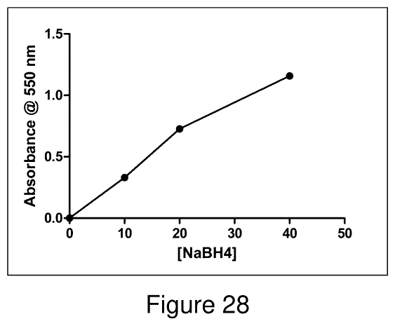

The synthesis of NiNPs was carried out at ice cold temperatures, allowing for a controlled and standard reaction. This was observed in the direct correlation between the concentration of NaBH₄ and the absorbance of the corresponding NiNP colloid at 550 nm (FIG. 28).

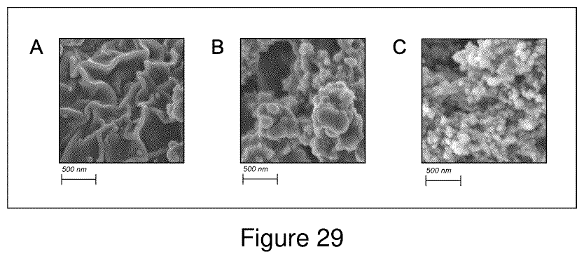

The colloids were further characterized by their physical properties, with particular focus on the nature of the synthesized nickel particles. Using Field emission scanning electron microscopy (FE-SEM), the nature of the particles formed (respective to the concentration of NaBH₄) were clearly observed (FIG. 29). At the highest NaBH₄ concentration (40 mM), it is clear that limited NiNPs are formed (FIG. 29A). Rather, there is a distinct degree of polymerization in the colloid, as seen by the sheet or polymer nature of the particles in the 40 mM NaBH₄ sample image. The degree of polymerization decreased as the concentration of NaBH₄ also decreased. The 20 mM NaBH₄ sample presented fewer polymeric molecules, but rather aggregates of spherical particles (FIG. 29B). The average size of these particles were approximately 250 nm in diameter, consequently too large to be deemed nanoparticles (diameter of 0-100 nm). The final sample, at an NaBH₄ concentration of 10 mM, presented aggregates of NiNPs, with an average particle diameter of approximately 50 nm, categorizing these as nanoparticles (FIG. 29C).

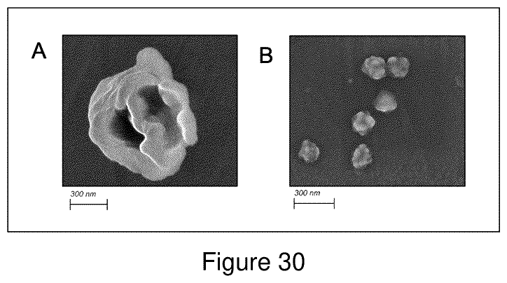

Although the synthesis of NiNPs was observed, the level of particle aggregation poses an issue for the immobilization of enzymes, as the surface area of the NiNPs is limited as aggregates (FIG. 29C). Using a combination of sonication and particle dispersion in ethanol (10% v/v ethylene glycol) directly after synthesis, the NiNPs were successfully dispersed, allowing for independent NiNPs to be found in the colloidal solution (FIG. 30).

Enzyme Immobilization

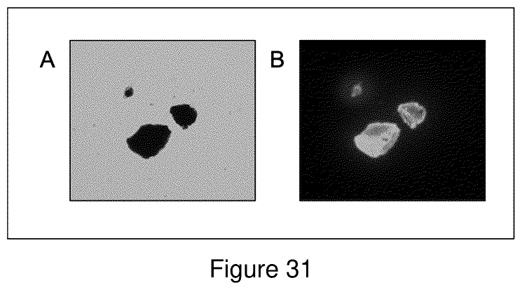

In order to provide putative evidence for the capability of our NiNPs for immobilization of enzymes, we used GFP due to its characteristic observed fluorescence when active. Following protein loading, as well as multiple washing processes, it was evident that GFP successfully, and with strong affinity, bound and was immobilized to the nickel particles (FIG. 31).

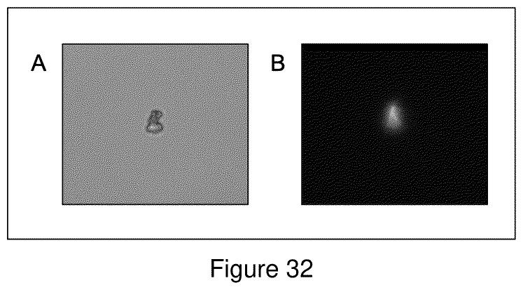

The same sample of GFP-NiNP as previously used (FIG. 31) was kept at room temperature for two weeks in order to observe the level of protein stability in unfavourable conditions over time. Following this, it was found that (although not to a matching level) the GFP retained a significant level of activity, as observed by the fluorescence of immobilized GFP to the nickel particles, even after two weeks at room temperature (FIG. 32).

1. An enzyme-polymer conjugate, comprising:
a histidine tagged enzyme, and
a cellulose matrix,
wherein the cellulose matrix is coated with a nickel-nanoparticle,
wherein the nickel-nanoparticle is prepared by combining a NaBH₄ solution and a NiCl solution; and
further wherein the histidine tagged enzyme is immobilized on the cellulose matrix through the formation of a coordinate covalent bond between the histidine tag and the nickel-nanoparticle.

2. The conjugate of claim 1, wherein the enzyme is selected from the group consisting of α-acetolactate, α-arabinosidase , α-galactosidase, a-rhamnosidase, β-galactosidase, β-glucanase, β-glucosidase, β-glucanase, β-mannanase, γ-lactamase, acetolactate decarboxylase, activase, adenosine deaminase, aminoacylase, aminopeptidase, amylase, amyloglucosidase, asparginase, aspartase, bromelain, carbonic anhydrase, catalase, cellulase, chitinase, chymosin, collagenase, cyclodextrinase, deoxyribonuclease I, dextranase, epimerase, esterase, formate dehydrogenase, galactinol synthase, glucanotransferase, glucoamylase, glucose isomerase, glucose oxidase, glutenase, hemicellulase, hexose oxidase, inulinase, invertase, laccase, lactase, lactate dehydrogenase, leucine dehydrogenase, levanase, lipase, lipoxygenase, lysozyme, methane monooxygenase, monoamine oxidase, muramidase, naphthalene dioxygenases, naphthalene monooxygenase, naringinase, nattokinase, nitrile hydratase, papain, pectinase, pectinesterase, penicillin G acylase, pentosanase, phenoloxidases, phenylalanine dehydrogenase, phytases, polyethylesterase, polygalacturonase, protease, protopectinase, pullulanase, raffinose synthase, rennet, sacrosidase, serratiopeptidase, sphingosine kinase, stachyose synthase, tannase, taxolase, thermolysin, transaminase, transaminase, transglutimases, trypsin, urease, xylanase, and xylose isomerase.

3. The conjugate of claim 1, wherein the cellulose matrix is cotton.

4. The conjugate of claim 3, wherein the cotton is cotton wool.

5. The conjugate of claim 1, wherein the histidine tag is a deca-histidine tag.

6. The conjugate of claim 1, wherein the histidine tag is on the N-terminal of the enzyme.

7. The conjugate of claim 1, wherein the histidine tag is on the C-terminal of the enzyme.

8. The conjugate of claim 1, wherein the enzyme catalytically converts a substrate into a product.

9. The conjugate of claim 1, wherein the immobilized enzyme has enhanced catalytic activity relative to an enzyme in free solution.

10. The conjugate of claim 1, wherein the NaBH₄ is a solution comprising 10 mM NaBH₄ in 0.2% w/v NaOH.

11. The conjugate of claim 1, wherein the NiCl is a solution comprising 5 mM NiCl.

12. A method of converting a substrate to a product, the method comprising contacting the substrate with the conjugate of claim 1.

13. A bioreactor comprising:
a fluid distribution chamber, having an inlet and an outlet,
a conjugate of claim 1, contained within the fluid distribution chamber,
wherein the histidine tagged enzyme is immobilized on the nickel nanoparticle cellulose matrix through the formation of a coordinate covalent bond between the histidine tag and a nickel nanoparticle bound to the cellulose matrix.

14. The bioreactor of claim 13, wherein a fluid containing a substrate is passed into the fluid distribution chamber through the inlet.

15. The bioreactor of claim 14, wherein the substrate is converted to a product by the enzyme.

16. The bioreactor of claim 15, wherein the product is recovered from the fluid distribution chamber from the outlet.

17. The bioreactor of claim 14, wherein the fluid contains a cofactor to enhance the activity of the histidine tagged enzyme.

18. The bioreactor of claim 17, wherein the cofactor is an organic or inorganic compound.

19. A method for producing a product by enzyme catalysis, the method comprising:
introducing a fluid containing a substrate to a bioreactor comprising a fluid distribution chamber,
wherein the fluid distribution chamber includes a conjugate of claim 1,
wherein the substrate is converted to a product by means of enzyme catalysis after coming into contact with the immobilized histidine tagged enzyme; and
recovering the product from the bioreactor.

20. The method of claim 19, wherein the product is recovered from the fluid distribution chamber from an outlet.

21. The method of claim 19, wherein the fluid contains a cofactor to enhance the activity of the histidine tagged enzyme.

22. The method of claim 21, wherein the cofactor is an organic or inorganic compound.


SEQUENCE LISTING (18 sequences)

SEQ ID NO: 1 - DNA, 725 bp, Artificial sequence
  Green fluorescent protein
atgagtaaaggagaagaacttttcactggagttgtcccaattcttgttgaattagatggtgatgttaatgggtacaaattttctgtcagtggagagggtgaaggtgatgcaacatacggaaaacttacccttaaatttatttgcactactggaaaactacctgttccatggccaacacttgtcactactctcacttatggtgttcaatgcttttcaagatatccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatgaccctgtccttttaccagacaaccattacctgtccacacaatctgccctttcgaaagatcccaacgaaaagagagaccacatggtccttcttgagtttgtaacggctgctgggattacacatggcatggatgaactatcaaaaccggttgggaa

SEQ ID NO: 2 - protein, 242 aa, Artificial sequence
  Green fluorescent protein
MSKGEELFTGVVPILVELDGDVNGYKFSVSGEGEGDATYGKLTLKFICTTGKLPVPWPTLVTTLTYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKTRHNIEDGGVQLADHYQQNTPIGDDPVLLPDNHYLSTQSALSKDPNEKRDHMVLLEFVTAAGITHGMDELYKTGWE

SEQ ID NO: 3 - DNA, 1266 bp, Artificial sequence
  Obtained from a metagenomic library
atgaccgataaacagaagaaattaccgtttcattttccgaaaaagttcttgtggggagcggcaacctcggcacaccaggtagaaggcggcaatcacaaccagtggagtgtatgggagcaggaaaatgccaaaactttagctgcaaaggcagattatcacaaggcagatttgccaaaatgggaagaaattaaaaaagaggcgaatagcccaaataattacctgtcaggtaaggcaacggatcattataacctctacgagaaggattttgacatcttaaaaaagatgaatatgaactcctttcgttttagcatagaatggtcgcgtatcgagccgaaagaaggtgtgtggaacgctgaggcgatccagcattataaagagtatataaaagctctcaagcaccgtgatattgagccggttatgaccttatttcatttcaccttacccgtgtggtttgctgagagaggtggctttgaaaagcgggcaaacgtcaagtattttgtgcgttttgcaaataaggttctgaaagatattggcacacatgtgcgctacatcgtcactattaacgagcctgaagtgtatgcaatgcaaagttaccaagagggcaattggccgccaaataaaacgagcaaggtagcaatgctgaaggtgtacaacaaccttgcggctgcacataacgaggttgccaagctggcgcataaaacgagccggcgttataaggtaggattggccaagaatgtcgcgtatcactatgcaggggacgacgcaaagctgactcgagcaactgccaaagcggcgcacgtaccagacgacatgttccttcgtaagataaagaagcacactgattggatagggttgaattattatttctcaaatcgttactacggctatcgggcgcataatccgaacgaaatgcagagtgatatgggctgggatatgcagccggagaatatccagcatgtacttgagcgactgtttgtaaaatacggcaagccgattattgtgacagaaaacggcgtggcagataaagacgatgaatatcgccaatggtggatagctaaaactttgcgagcgatggacgcagcgctacagaatggcgtgccgcttgaggggtatttgcactggagtttgctggataattttgaatgggcagagggtaagtggccacgttttggcttgcttgaggtggattatgacagtagtcagcgaaaaccacgtaaaagtgcggtgtggtttgcccgtgttttaaagcatctacgtgatgtataa

SEQ ID NO: 4 - protein, 421 aa, Artificial sequence
  Obtained from a metagenomic library
MTDKQKKLPFHFPKKFLWGAATSAHQVEGGNHNQWSVWEQENAKTLAAKADYHKADLPKWEEIKKEANSPNNYLSGKATDHYNLYEKDFDILKKMNMNSFRFSIEWSRIEPKEGVWNAEAIQHYKEYIKALKHRDIEPVMTLFHFTLPVWFAERGGFEKRANVKYFVRFANKVLKDIGTHVRYIVTINEPEVYAMQSYQEGNWPPNKTSKVAMLKVYNNLAAAHNEVAKLAHKTSRRYKVGLAKNVAYHYAGDDAKLTRATAKAAHVPDDMFLRKIKKHTDWIGLNYYFSNRYYGYRAHNPNEMQSDMGWDMQPENIQHVLERLFVKYGKPIIVTENGVADKDDEYRQWWIAKTLRAMDAALQNGVPLEGYLHWSLLDNFEWAEGKWPRFGLLEVDYDSSQRKPRKSAVWFARVLKHLRDV

SEQ ID NO: 5 - DNA, 963 bp, Pigeon pea
atggcccctgatatgatcagcaccctcactgatgctcaagctaaggccagcggcggtcttggccgtgcctacgtcaccttcctcgccggtaacggcgactacgtgaaaggtgttgttggcttggccaaaggtctgaggaaggtgaagagcatgtaccctttggtggttgcagtgttacccgatgttccccaagagcaccgcaacattctcatcactcagggttgcattgttagagagattgagcctgtgtacccccctaagaaccaaacacagtttgccatggcatattacgtcatcaactattccaagctacgtatttgggagtttgtggagtatagtaagatgatttacctagacggtgatatccaagtttttagcaacattgaccacttgtttgatttgcctgatgattacttctacgcggtgatggactgtttctgcgagcccacttgggcccacactccgcagtttcagatcggttactgccagcagtgccctgataaggttcagtggccctctgattttgggcccaagcctcctctctatttcaatgctggcatgtttgtttatgtgcccaatttggatacctaccatgatcttcttcaaacagtccaagtcaccaagcccacttcctttgctgaacaagattttttgaacatgtacttcaaggacaaatataggcccataccaaatgtctacaatcttgtgctggccatgctgtggcgtcaccctgagaatgttgagcttgacaaagtcaaagtggttcactattgtgctgctggatctaagccttggaggtacactgggaaagaagagaacatggatagagaagatattaagatgttggtgaagaagtggtgggatatatatgaggatgagaatttggactacaaaaatcccctcactgcggcgcttttggaggctggtggatttaagtttgttcctgccccttcggccgcttag

SEQ ID NO: 6 - protein, 320 aa, Pigeon pea
MAPDMISTLTDAQAKASGGLGRAYVTFLAGNGDYVKGVVGLAKGLRKVKSMYPLVVAVLPDVPQEHRNILITQGCIVREIEPVYPPKNQTQFAMAYYVINYSKLRIWEFVEYSKMIYLDGDIQVFSNIDHLFDLPDDYFYAVMDCFCEPTWAHTPQFQIGYCQQCPDKVQWPSDFGPKPPLYFNAGMFVYVPNLDTYHDLLQTVQVTKPTSFAEQDFLNMYFKDKYRPIPNVYNLVLAMLWRHPENVELDKVKVVHYCAAGSKPWRYTGKEENMDREDIKMLVKKWWDIYEDENLDYKNPLTAALLEAGGFKFVPAPSAA

SEQ ID NO: 7 - DNA, 2051 bp, Pigeon pea
atgagcctattccgcttcaaggtgtggtggaccacccactgggccggaagcaacgggcacgagatggagcacgaaacccaaatgctcttgcttgacaaaaaccatgcactggggcgcccctacgtgttgcttctccccatccttgaaggctccttccgagcctcgctccaacccggcttcgatgactacgtggacgtctgcgtggagagcgggtccacacgtgtcaccagctccagcttcaggagctgcttgtacatccacgtgggcgacgacccctacaggcttgtcaaagaagcaaccaaagtggttagaatgcatctggggactttcaagcttctggaagaaaaaaccccaccagtgattgtggacaagttcggttggtgcacgtgggacgcattttacttgaaggtgcatccggagggagtatgggaaggggtgaaggggttggtggagggagggtgtccgccggggatggtcctcatcgacgacgggtggcaatcggttagtcacgacgaggaggaagaagaggctgtggaccgaacctcagctggggaccaaatgccttgcaggctcgtgaggttggaggagaatgacaagttcaggaattacaggagtggagggaggaatgggtatgggatgggggcgtttgttagggatttgaaggagcggtttgggagcgtggagcaggtgtacgtttggcacgcgctctgtgggtactggggtggtgtcaggcctcaggctccgggcatgcccgaagctaaggtgataactccgaagctgtccaaagggttgggaatgacaatggaggatttggcggtggataagattgtgaataacggcgtgggactagtgccacctcacctcgctcaccgcttgttccaggggattcactcgcgtttggaatccgccggaattgacggcgtcaagcttgacgttatacacttgcttgagatgctagcggaggaatacggtgggcgtgttgagttagccaaagcttattacaaagcacttactgcttcggtgaagaagcatttcaaaggcaacggagtcattgccagcatggagcattgtaatgacttctttctccttggcaccgaagccatatcccttgggcgcgtaggagatgatttttggtgcactgatccgtctggggatccaaatggtacgtattggctccaaggatgtcacatggtgcactgtgcctacaacagcttgtggatgggaaatttcattcagccggattgggacatgttccagtccactcacccttgtgccgaattccatgctgcctctagggccatatctggtggacccatttacgttagcgactgtgttggaaagcacaacttcaagttgctcaagaccctcgctttgcctgatggctccatcttgcgttgtcaacattatgcacttcccacacgagactctttatttgaagatccattacatgatggcaaaacaatgctcaaaatttggaatctcaacaaatatacaggtgttttgggagtatttaattgccaggaggtgggtggtgccctgtgactcggcgaaacaagagtgcctctgaattttcacgcactgtgacatgcttagcgagtcctggggatattgaatggagcaacgggaaaagtccaatagacataaaagaagtggaagtgtttgctctatatttgctcaaggacaacaaactaaagctcatgaagtcttcggagaacttggaggtttcactggagccatttagttttgagcttttgacagtgtctccagtgacagtgttaccaaaaaggttaattcagtttgctccaattggactagtgaacatgcttaactcgggtggtgccattcagtccctggagtttgatgaccacacggatttggtcaaaattggggtgaggggttgtggggagatgagggtgtttgcttcagagaagccacttagttgcacaattgatggggtggctgtgaaatttgattacgaggataagatggtgagggtgcaagttccatggcctagttcttcaaaattgtcaatgcttgagtttttgttttaa

SEQ ID NO: 8 - protein, 683 aa, Pigeon pea
MSLFRFKVWWTTHWAGSNGHEMEHETQMLLLDKNHALGRPYVLLLPILEGSFRASLQPGFDDYVDVCVESGSTRVTSSSFRSCLYIHVGDDPYRLVKEATKVVRMHLGTFKLLEEKTPPVIVDKFGWCTWDAFYLKVHPEGVWEGVKGLVEGGCPPGMVLIDDGWQSVSHDEEEEEAVDRTSAGDQMPCRLVRLEENDKFRNYRSGGRNGYGMGAFVRDLKERFGSVEQVYVWHALCGYWGGVRPQAPGMPEAKVITPKLSKGLGMTMEDLAVDKIVNNGVGLVPPHLAHRLFQGIHSRLESAGIDGVKLDVIHLLEMLAEEYGGRVELAKAYYKALTASVKKHFKGNGVIASMEHCNDFFLLGTEAISLGRVGDDFWCTDPSGDPNGTYWLQGCHMVHCAYNSLWMGNFIQPDWDMFQSTHPCAEFHAASRAISGGPIYVSDCVGKHNFKLLKTLALPDGSILRCQHYALPTRDSLFEDPLHDGKTMLKIWNLNKYTGVLGVFNCQGGGWCPVTRRNKSASEFSRTVTCLASPGDIEWSNGKSPIDIKEVEVFALYLLKDNKLKLMKSSENLEVSLEPFSFELLTVSPVTVLPKRLIQFAPIGLVNMLNSGGAIQSLEFDDHTDLVKIGVRGCGEMRVFASEKPLSCTIDGVAVKFDYEDKMVRVQVPWPSSSKLSMLEFLF

SEQ ID NO: 9 - DNA, 2553 bp, Streptococcus mutans
atggaacagtcaaataggcaaacggctgaaccagctattaggtcagctgaaacggtggattcgaccattaactcttttcaagagacagaccttaaggtgcaagagaaggaggatgttgcggctgcagtacagacagaatcagcgtcaatagattctaatgaacagggacaatcggtctctgcaaatactaacacacaatctcaagcgaagaaactttctaacaattcccatcaggagccaatgcaaatggtatctgccgccaataaagaaagggcagtgctagaaactgcacagaaccaaaagaatggcaacatgataaatctgacaacagataaagcagtctatcaggcgggagaggctgttcatttgaaccttactttaaacaatacaacatctttagcccaaaatattacagctactgctgaggtttattctcttgaaaataaattaaagacacttcagtatacgaagtatcttctgcctaatgaaagttatacaactcaaaaaggtgaattcgttattcctgcaaactccttagctaataatcgcggttatcttttgaaggttaacatatcagatagccaaaataatattttagagcagggcaatcgggctattgcggttgaggatgactggcgtacctttccgcgttatgctgctattggaggttctcaaaaagacaataacagtgtcttgactaagaacttaccagattattatcgcgaattagagcagatgaaaaatatgaacattaattcctatttcttctatgatgtttataagtctgctacaaatcctttccctaatgttcctaagtttgatcagtcttggaattggtggagccattcgcaggttgaaacagatgctgttaaagccttggtcaatcgtgtccatcaaactggcgctgttgccatgctctataatatgattttagcacagaatgctaatgaaacggctgttttaccagatactgagtacatctataattatgagactggtggttatggtcaaaatggtcaggtcatgacttactctattgatgataagccgctgcaatattattacaatcctttgagtaaaagttggcaaaattatatttctaatgccatggctcaagctatgaaaaatggcggttttgatggctggcagggagatacaattggagataatcgtgttctttcccataatcaaaaggacagtcgagatattgctcattcctttatgttatctgatgtctatgctgaatttctcaataaaatgaaggaaaaactgcctcagtattatttaacactcaatgatgttaatggtgaaaatatcagcaaactcgccaacagcaaacaagatgtgatttacaatgaattatggccttttggaacttcagctttggggaaccgtccccaagaaagttatggtgacttgaaagctcgtgttgatcaagttcgccaagcgacagggaaatctttgattgtcggagcttatatggaagagcctaaatttgatgataatagggttcctctcaatggtgcagcgcgtgacgttttagcttcagcaacttaccaaacagatgcggttctgctgacaactgcggccattgcggcagcaggaggatatcacatgtctctggctgctctggctaatcctaatgatgggggtggtgtcggtgtcttagaaacagcttattatccaacacaaagcctcaaggtttcgaaagagctcaatcgtaaaaactatcattaccaacaatttattacggcttatgaaaatcttttgcgtgataaagttgaaaatgattctgctgaacctcagactttcactgctaacggtcggcagctatcgcaagatgctttggggatcaatggcgatcaggtttggacttatgccaaaaagggaaacgatttcagaacgattcaattgctcaaccttatgggaattacatccgactggaaaaatgaagatggttatgaaaataataaaacacctgatgagcaaaccaatttattggttacttatcctttgactggtgtgtctatggcagaggctgatcgaatagctaaacaagtctatctgacgtcaccagatgattggctgcaatctagtatgatttctctagcgacccagataaaaacgaatgagaatggcgatcctgttctttatattcaagtgccaagactgacgctttgggatatgatttatattaatgaaaccattaaaccagaaacgcctaaagttccagaacagccccaacatcctgctaggacacttgaaccagcaattccgcaaactccagaagcagtcagccctctcccagtagctaataagcaggcagtagatgaaaataaaaatgagattgtttcagccttaaccggtgaagaaaatgacttgcagttgccaactctttccaaacgatcattgtcaatctcccaagcagagttaccgcaaacaggagataacaatgaaacgcgctccaatctcctcaaagtgataggtgctggtgcgcttctaatcggcgctgcaggattattaagcttgataaagggtagaaaaaaagattga

SEQ ID NO: 10 - protein, 850 aa, Streptococcus mutans
MEQSNRQTAEPAIRSAETVDSTINSFQETDLKVQEKEDVAAAVQTESASIDSNEQGQSVSANTNTQSQAKKLSNNSHQEPMQMVSAANKERAVLETAQNQKNGNMINLTTDKAVYQAGEAVHLNLTLNNTTSLAQNITATAEVYSLENKLKTLQYTKYLLPNESYTTQKGEFVIPANSLANNRGYLLKVNISDSQNNILEQGNRAIAVEDDWRTFPRYAAIGGSQKDNNSVLTKNLPDYYRELEQMKNMNINSYFFYDVYKSATNPFPNVPKFDQSWNWWSHSQVETDAVKALVNRVHQTGAVAMLYNMILAQNANETAVLPDTEYIYNYETGGYGQNGQVMTYSIDDKPLQYYYNPLSKSWQNYISNAMAQAMKNGGFDGWQGDTIGDNRVLSHNQKDSRDIAHSFMLSDVYAEFLNKMKEKLPQYYLTLNDVNGENISKLANSKQDVIYNELWPFGTSALGNRPQESYGDLKARVDQVRQATGKSLIVGAYMEEPKFDDNRVPLNGAARDVLASATYQTDAVLLTTAAIAAAGGYHMSLAALANPNDGGGVGVLETAYYPTQSLKVSKELNRKNYHYQQFITAYENLLRDKVENDSAEPQTFTANGRQLSQDALGINGDQVWTYAKKGNDFRTIQLLNLMGITSDWKNEDGYENNKTPDEQTNLLVTYPLTGVSMAEADRIAKQVYLTSPDDWLQSSMISLATQIKTNENGDPVLYIQVPRLTLWDMIYINETIKPETPKVPEQPQHPARTLEPAIPQTPEAVSPLPVANKQAVDENKNEIVSALTGEENDLQLPTLSKRSLSISQAELPQTGDNNETRSNLLKVIGAGALLIGAAGLLSLIKGRKKD

SEQ ID NO: 11 - DNA, 1539 bp, Bacillus amyloliquefaciens
atggcacttgaaaaatttgcagatgaactgccgattatcgaaacactgaagccgcagaagacatcaaacggcagcacgtattatgaagtcacgatgaaggaatgctttcacaagctgcaccgtgatctcccgccgacccggctgtggggctataacggtttgtttcccggcccgacgatcgacgtgaaccaagatgagaacgtctatattaaatggatgaatgacctgccggataagcattttctccctgtggaccataccattcaccattcagagggcggccatcaggaacccgacgtcaaaaccgtcgtccatttacacggaggagcaacgccgccggacagcgacggctatccggaagcctggttcacacgggatttcaaggagaaggggccttattttgaaaaagaggtataccactatccaaacaaacagcgcggggcgctattatggtatcacgaccacgccatggcaattacgaggctcaatgtgtacgccgggcttgccggcatgtatatcatccgcgagcgaaaagaaaagcagctgaagcttcccgccggagaatacgacgtaccgcttatgattatggaccgcacgttaaatgacgacggttccttgttttatccgagcgggcccgataatccttccgaaacgctgccgaatccttcaatcgttccattcctttgcggaaataccattctcgtcaacggcaaagcgtggccgtatatggaagtcgaaccgcggacatatcgtttccgtatccttaacgcctcaaatacgagaacattttccctctcgctcaataatggcggccggtttattcaaatcggttctgacggcggactgctcccccgttctgtcaagacacagtccatcagcttagccccggctgagcggtatgatgtgctcattgatttctccgcttttgacggagaacatattattttaacgaacggcaccggctgcgggggcgacgtcaatccggataccgacgccaatgtgatgcaattccgcgtcacaaaaccgctgaagggagaagacaccagccggaagcctaaatatctgtcagccatgcctgatatgacatcaaaaagaatacacaatatcaggacgcttaaactcacaaacacgcaagacaaatacggccggccggttttaacactcaataacaagcgctggcatgatcccgtgacagaagcgccgcggctcggctcaacggaaatctggtcgattatcaacccgacgcggggaacccatccgatacacctgcacttggtttccttccaagtccttgaccggcgtccttttgacttagaacgttataacaaattcggcgacattgtgtatacaggccccgccgtcccgccgcctccaagtgaaaaaggctggaaagacaccgtgcaggcgcactccggagaagtcatcagaatcgcggcgacattcgcgccttacagcggacggtacgtatggcattgtcatattttagaacatgaagattatgacatgatgagaccgatggacgtcacagaaaagcagtaa

SEQ ID NO: 12 - protein, 512 aa, Bacillus amyloliquefaciens
MALEKFADELPIIETLKPQKTSNGSTYYEVTMKECFHKLHRDLPPTRLWGYNGLFPGPTIDVNQDENVYIKWMNDLPDKHFLPVDHTIHHSEGGHQEPDVKTVVHLHGGATPPDSDGYPEAWFTRDFKEKGPYFEKEVYHYPNKQRGALLWYHDHAMAITRLNVYAGLAGMYIIRERKEKQLKLPAGEYDVPLMIMDRTLNDDGSLFYPSGPDNPSETLPNPSIVPFLCGNTILVNGKAWPYMEVEPRTYRFRILNASNTRTFSLSLNNGGRFIQIGSDGGLLPRSVKTQSISLAPAERYDVLIDFSAFDGEHIILTNGTGCGGDVNPDTDANVMQFRVTKPLKGEDTSRKPKYLSAMPDMTSKRIHNIRTLKLTNTQDKYGRPVLTLNNKRWHDPVTEAPRLGSTEIWSIINPTRGTHPIHLHLVSFQVLDRRPFDLERYNKFGDIVYTGPAVPPPPSEKGWKDTVQAHSGEVIRIAATFAPYSGRYVWHCHILEHEDYDMMRPMDVTEKQ

SEQ ID NO: 13 - DNA, 663 bp, Escherichia coli
atgaaagacatagatacactcatcagcaacaatgcactatggtcaaaaatgctggtggaagaggatcccgggttttttgagaaactggcacaagcgcaaaaaccgcgctttctatggattggatgttccgacagtcgcgttcctgcagaacgtttaaccggtcttgagccgggcgaactctttgttcaccgtaatgttgctaacctggtcattcacactgacctgaactgcctttccgtggttcagtatgcagtggatgtactcgaagttgaacacattattatctgtggccactacggttgcggcggcgtacaagccgcagttgaaaacccggaactggggcttatcaacaactggctgctgcatatccgcgatatctggttcaaacatagctcattgctcggcgaaatgccgcaagagcgccgtctggataccttgtgtgaactgaacgtcatggaacaggtgtataacctgggccactccaccattatgcaatcagcgtggaaacgcgggcagaaagttaccattcacggctgggcctacggcattcacgacggcttgctgcgtgatctggatgttaccgccaccaaccgcgaaacccttgagcaacgttaccgtcacgggatttccaacctcaagctgaaacacgccaaccacaaataa

SEQ ID NO: 14 - protein, 220 aa, Escherichia coli
MKDIDTLISNNALWSKMLVEEDPGFFEKLAQAQKPRFLWIGCSDSRVPAERLTGLEPGELFVHRNVANLVIHTDLNCLSVVQYAVDVLEVEHIIICGHYGCGGVQAAVENPELGLINNWLLHIRDIWFKHSSLLGEMPQERRLDTLCELNVMEQVYNLGHSTIMQSAWKRGQKVTIHGWAYGIHDGLLRDLDVTATNRETLEQRYRHGISNLKLKHANHK

SEQ ID NO: 15 - DNA, 2262 bp, Escherichia coli
atgtcgcaacataacgaaaagaacccacatcagcaccagtcaccactacacgattccagcgaagcgaaaccggggatggactcactggcacctgaggacggctctcatcgtccagcggctgaaccaacaccgccaggtgcacaacctaccgccccagggagcctgaaagcccctgatacgcgtaacgaaaaacttaattctctggaagacgtacgcaaaggcagtgaaaattatgcgctgaccactaatcagggcgtgcgcatcgccgacgatcaaaactcactgcgtgccggtagccgtggtccaacgctgctggaagattttattctgcgcgagaaaatcacccactttgaccatgagcgcattccggaacgtattgttcatgcacgcggatcagccgctcacggttatttccagccatataaaagcttaagcgatattaccaaagcggatttcctctcagatccgaacaaaatcaccccagtatttgtacgtttctctaccgttcagggtggtgctggctctgctgataccgtgcgtgatatccgtggctttgccaccaagttctataccgaagagggtatttttgacctcgttggcaataacacgccaatcttctttatccaggatgcgcataaattccccgattttgttcatgcggtaaaaccagaaccgcactgggcaattccacaagggcaaagtgcccacgatactttctgggattatgtttctctgcaacctgaaactctgcacaacgtgatgtgggcgatgtcggatcgcggcatcccccgcagttaccgcaccatggaaggcttcggtattcacaccttccgcctgattaatgccgaagggaaggcaacgtttgtacgtttccactggaaaccactggcaggtaaagcctcactcgtttgggatgaagcacaaaaactcaccggacgtgacccggacttccaccgccgcgagttgtgggaagccattgaagcaggcgattttccggaatacgaactgggcttccagttgattcctgaagaagatgaattcaagttcgacttcgatcttctcgatccaaccaaacttatcccggaagaactggtgcccgttcagcgtgtcggcaaaatggtgctcaatcgcaacccggataacttctttgctgaaaacgaacaggcggctttccatcctgggcatatcgtgccgggactggacttcaccaacgatccgctgttgcagggacgtttgttctcctataccgatacacaaatcagtcgtcttggtgggccgaatttccatgagattccgattaaccgtccgacctgcccttaccataatttccagcgtgacggcatgcatcgcatggggatcgacactaacccggcgaattacgaaccgaactcgattaacgataactggccgcgcgaaacaccgccggggccgaaacgcggcggttttgaatcataccaggagcgcgtggaaggcaataaagttcgcgagcgcagcccatcgtttggcgaatattattcccatccgcgtctgttctggctaagtcagacgccatttgagcagcgccatattgtcgatggtttcagttttgagttaagcaaagtcgttcgtccgtatattcgtgagcgcgttgttgaccagctggcgcatattgatctcactctggcccaggcggtggcgaaaaatctcggtatcgaactgactgacgaccagctgaatatcaccccacctccggacgtcaacggtctgaaaaaggatccatccttaagtttgtacgccattcctgacggtgatgtgaaaggtcgcgtggtagcgattttacttaatgatgaagtgagatcggcagaccttctggccattctcaaggcgctgaaggccaaaggcgttcatgccaaactgctctactcccgaatgggtgaagtgactgcggatgacggtacggtgttgcctatagccgctacctttgccggtgcaccttcgctgacggtcgatgcggtcattgtcccttgcggcaatatcgcggatatcgctgacaacggcgatgccaactactacctgatggaagcctacaaacaccttaaaccgattgcgctggcgggtgacgcgcgcaagtttaaagcaacaatcaagatcgctgaccagggtgaagaagggattgtggaagctgacagcgctgacggtagttttatggatgaactgctaacgctgatggcagcacaccgcgtgtggtcacgcattcctaagattgacaaaattcctgcctga

SEQ ID NO: 16 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 17 - DNA, 477 bp, Bacteriophage lambda
atggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatga

SEQ ID NO: 18 - protein, 158 aa, Bacteriophage lambda
MVEINNQRKAFLDMLAWSEGTDNGRQKTRNHGYDVIVGGELFTDYSDHPRKLVTLNPKLKSTGAGRYQLLSRWWDAYRKQLGLKDFSPKSQDAVALQQIKERGALPMIDRGDIRQAIDRCSNIWASLPGAGYGQFEHKADSLIAKFKEAGGTVREIDV